(19) United States
(12) Patent Application Publication
(10) Pub. No.: US 2009/0035228 A1
(43) Pub. Date: Feb. 5, 2009

(54) SKIN AND SURFACE DISINFECTANT COMPOSITIONS CONTAINING BOTANICALS

(76) Inventors: Shanta Modak, River Edge, NJ (US); Nayana Baiju, New York, NY (US); Lauserpina Caraos, Hollis, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/016,788

(22) Filed: Jan. 18, 2008

Related U.S. Application Data
(60) Provisional application No. 60/953,654, filed on Aug. 2, 2007.

(51) Int. Cl. A61K 8/36; A61K 31/19; A61Q 11/00; A61Q 15/00; A61Q 17/04; A61Q 99/00; A61P 17/10; A61P 17/00; A01P 1/00; C11D 3/48

(52) U.S. Cl. 424/49; 424/65; 424/59; 514/558; 510/119; 510/130; 510/138; 510/131; 510/160; 510/382

(57) ABSTRACT

The present invention relates to a skin or surface disinfectant composition with broad spectrum antimicrobial activity comprising one or more essential oil (and/or one or more component thereof) and one or more fruit acid. The compositions of the invention may be used as non-toxic alternatives to conventional disinfectants or may be added to other antimicrobial agents to enhance their activity. The invention provides effective alternatives to harsher products which may be particularly useful in personal care and household products and where children and/or pet exposure may be a concern.

### PRIORITY CLAIMED

This application claims priority benefits to U.S. Provisional Application Ser. No. 60/953,654, filed Aug. 2, 2007, which is hereby incorporated by reference in its entirety herein.

### GRANT INFORMATION

Not applicable.

### 1. INTRODUCTION

The present invention relates to a skin or surface disinfectant composition with broad spectrum antimicrobial activity comprising one or more essential oil (and/or one or more component thereof) and one or more fruit acid. The compositions of the invention may be used as non-toxic alternatives to conventional disinfectants or may be combined with other antimicrobial agents to enhance their activity. The invention provides effective alternatives to harsher products, and may be particularly useful in personal care and household product applications and where children and/or pet exposure may be a concern.

### 2. BACKGROUND OF THE INVENTION

Essential oils are volatile oils obtained from plant or animal sources and are composed of complex mixtures of several constituents, such as monoterpenes and sesquiterpene hydrocarbons, monoterpene and sesquiterpene alcohols, esters, ethers, aldehydes, ketones, oxides and the like. These essential oils and their isolated constituents are frequently utilized as fragrance and flavor agents, and have been widely used in folk medicine for wound healing properties.

Scientific research has corroborated the beneficial effects of essential oils. Essential oils of eucalyptus have been found to “possess central and peripheral analgesic effects as well as neutrophil-dependent and independent anti-inflammatory activities” (Silva et al., 2003, J. Ethnopharmacol. 89(2-3);277-283), and similar activity has been observed in essential oils from Lavendula angustifolia Mill. (Hajhashemi et al., 2003, J. Ethnopharmacol. 89(1):67-71). Essential oils have been demonstrated to exhibit antibacterial (Bezic et al., 2003, Phytother. Res. 17(9:1037-1040; Goren et al., 2003, Z. Naturforsch. 58(9-10):687-690; de Abreu Gonzaga et al., 2003, Planta Med. 69(8:773-775; Valero and Salmera, 2003, Int. J. Food Microbiol. 85(1-2): 73-81) and antifungal (Paranagama et al., 2003, Lett. Appl. Microbiol. 37(1):86-90; Shin, 2003, Arch. Pharm. Res. 26(5):389-393; Velluti et al., 2003, Int. J. Food Microbiol. 89:145-154) activities. Virucidal activity of essential oils has also been observed, including direct virucidal effects against Herpes simplex viruses types 1 and 2 (Garcia et al., Phytother. Res. 17(9):1073-1075; Minami et al., 2003, Microbial Immunol. 47(a):681-684; Schuhmacher et al., 2003, Phytomedicine 10:504-510).

United States Patent Application Publication No. 20050048139 by Modak et al., published Mar. 3, 2005, relates to topical compositions comprising an emollient solvent and an essential oil, which may further comprise additional additives, among which citric acid, glycolic acid and lactic acid are cited. It does not recognize the synergistic activity between essential oils and fruit acids nor does it disclose the concentrations of fruit acids to be used to provide a synergistic effect.

United States Patent Application Publication No. 20050019431 by Modak et al., published Jan. 27, 2005, relates to compositions comprising a quaternary ammonium compound and an essential oil (or active component thereof).

A number of patent applications relate to compositions comprising an essential oil (or component thereof) where zinc salts are added to inhibit irritation associated with essential oils. Examples of such patent applications include United States Patent Application Publication No. 20040102429 by Modak et al., published May 27, 2004 and United States Patent Application Publication No. 20050238602 by Modak et al., published Oct. 27, 2005.

U.S. Pat. No. 6,858,317 by Aamodt et al., issued Feb. 22, 2005, relates to methods for protecting wood from mold and sapstaining fungi which employ a non-toxic mold inhibitor which may be a plant extract such as an essential oil.

U.S. Pat. No. 5,100,652 by Kross et al., issued Mar. 31, 1992, relates to low concentration chlorous-acid generating oral hygience compositions which may comprise an essential oil as a flavoring agent.

U.S. Pat. No. 5,310,546 by Douglas, issued May 10, 1994, relates to a mouthrinse preparation comprising hydrogen peroxide, zinc chloride, sodium citrate, sodium lauryl sulfate, citric acid and ethanol and optionally an essential oil which is a denaturing agent.

BiON offers several skin care products comprising citric acid, botanicals, and other agents for topical use (San Diego, Calif., US).

Johnson et al. (U.S. Pat. No. 6,319,958 and US20020165130) relates to the use of sesquiterpenoids to promote uptake of exogenous antimicrobial compounds. Similarly, a related article discloses the use of sesquiterpenoids, such as nerolidol, famesol, bisabolol and apritone, in enhancing bacterial permeability and susceptibility to exogenous antimicrobial compounds, suggesting that sesquiterpenoids have a non-specific and general effect (Brehm-Stecher et al. 2003, Antimicrobial Agents and Chemotherapy, 47(10):3357-3360). In particular, Brehm-Stecher et al. report that nerolidol, farnesol, bisabolol and apritone enhanced the susceptibility of S. aureus to the antibiotics erythromycin, gentamicin, vancomycin, ciproflaxin, clindamycin, and tetracycline.

U.S. Pat. No. 4,867,898 by Spaulding et al., issued Sep. 19, 1989, relates to a liquid hard surface cleaner comprising pine oil and organic, oil-soluble acids at a pH from 0-6.

U.S. Pat. No. 6,753,305 by Raso and Caselli, issued Jun. 22, 2004, relates to a hard surface disinfectant comprising up to 20 percent of cinnamon oil or a component thereof, 0.01-5 percent of an organic acid, and optionally an additional essential oil.

International Patent Application Publication No. WO2007077573 by Mukhopadhyay, published Jul. 12, 2007, relates to antimicrobial compositions comprising an antimicrobial agent, such as triclosan, and a functionalized hydrocarbon, where the functionalized hydrocarbon can be an essential oil, and/or a solvent.

There is a continuing desire for an antimicrobial composition that is non-irritating, safe, and effective for repeated use in various professional and non-professional settings.

### 3. SUMMARY OF THE INVENTION

The present invention relates to a skin or surface disinfectant composition with broad spectrum antimicrobial activity comprising one or more essential oil (and/or one or more component (i.e., an “Individual Constituent” or “IC”) thereof) and one or more fruit acid. It is based, at least in part, on the discovery that a combination of an essential oil or component thereof together with a fruit acid can confer superior antimicrobial properties on personal care, veterinary, as well as household products. In preferred, non-limiting embodiments, the compositions of the invention further comprise up to about 20 percent alcohol, which facilitates the solubilization of the essential oil(s)/IC(s) and fruit acid.

In various non-limiting embodiments, the present invention may be utilized in personal care products such as soaps, scrubs, cosmetics, creams and lotions and veterinary products such as pet shampoos and pet cleansing wipes. In other non-limiting embodiments, the present invention may be utilized in household products such as general purpose cleaning fluids, spray cleaners, laundry detergents, food washes, etc.

The compositions of the invention may be used as non-toxic alternatives to conventional disinfectants or may be added to other antimicrobial agents to enhance their activity. The invention provides effective alternatives to harsher products which may be particularly useful in personal care and household products and where children and/or pet exposure may be a concern.

### 4. DETAILED DESCRIPTION OF THE INVENTION

For clarity of description, and not by way of limitation, the detailed description of the invention is divided into the following subsections:

(i) essential oils;

(ii) fruit acids;

(iii) combinations of essential oils/ICs and fruit acids;

(iv) personal care products;

(v) veterinary products; and

(vi) household/industrial products.

### 4.1 Essential Oils

Essential oils (“EOs”), as defined herein, are volatile oils obtained from plant or animal sources, or their synthetic equivalents, and are composed of complex mixtures of several constituents as monoterpenes and sesquiterpene hydrocarbons, monoterpene and sesquiterpene alcohols, esters, ethers, aldehydes, ketones, oxides and the like. Examples of EOs include, but are not limited to, cinnamon oil, basil oil, bergamot oil, clary sage oil, ylang-ylang oil, neroli oil, sandalwood oil, frankincense oil, ginger oil, peppermint oil, lavender oil, jasmine absolute, geranium oil bourbon, spearmint oil, clove oil, patchouli oil, rosemary oil, rosewood oil, sandalwood oil, tea tree oil, vanilla oil, lemongrass oil, cedarwood oil, balsam oils, tangerine oil, Hinoki oil, Hiba oil, ginko oil, eucalyptus oil, lemon oil, orange oil, and sweet orange oil. In preferred non-limiting embodiments of the invention, the EO is selected from one or more EO from the group consisting of cinnamon oil (bark or leaf), lemograss oil, citonella oil, basil oil, and orange oil.

Individual constituents (“ICs”) of essential oils may be isolated from the oil (natural) or entirely or partially synthetic, and include, but are not limited to, 1-citronellol, α-amylcinnamaldehyde, lyral, geraniol, farnesol, hydroxycitronellal, isoeugenol, eugenol, eucalyptol, linalool, citral, thymol, limonene and menthol. Further examples of ICs include sesquiterpenoid compounds, which may be the active compounds in the essential oils. Sesquiterpenoid compounds, containing 15 carbons, are formed biosynthetically from three 5-carbon isoprene units. Sesquiterpenoid compounds include, but are not limited to, farnesol, nerolidol, bisabolol, apritone, chamazulene, santalol, zingiberol, carotol, and caryophyllen. Mixtures of one or more EO, one or more IC, and one or more EO as well as one or more IC, are encompassed by the present invention. In specific non-limiting embodiments of the invention, an IC is selected from the (non-limiting) group consisting of camphor, alpha-pinene, constituents of cinnamon leaf oil such as, cinnamaldehyde, cinnamylacetic ester, cinnamic acid, ethyl cinnamate, methyl chavicol, linalool, beta-caryophyllene, and eugenol; constituents of lemongrass oil such as d-limonene, geranyl acetate, nerol, geraniol, citral, and/or myrcene; constituents of citronella oil such as geraniol, citronellol, citronellal, geranyl acetate, limonene, methyl isoueugenol, and/or elemol; components of basil oil such as camphor, limonene, and/or β-selinene; and constituents of orange oil such as a-pinene, sabinene, myrcene, limonene, linalool, citronellal, neral and/or geranial.

An EO or IC for use in the invention may be obtained from its natural source or may be chemically synthesized.

### 4.2 Fruit Acids

Fruit acids which may be used according to the invention include but are not limited to citric acid, glycolic acid, lactic acid, malic acid, tartaric acid and acetic acid. In preferred non-limiting embodiments of the invention, the fruit acid is citric acid.

A fruit acid for use in the invention may be obtained from its natural source or may be chemically synthesized.

### 4.3 Combinations of Essential Oils/ICs and Fruit Acids

The present invention provides for compositions comprising a combination of one or more essential oil (and/or one or more IC thereof) and one or more fruit acid. Preferably, this combination produces a synergistic anti-microbial effect against at least one microbe selected from the group consisting of Escherichia coli, Pseudomonas aeruginosa, Staphylococcus aureus, methicillin-resistant S. aureus, and Candida albicans (“synergistic” means that the antimicrobial effect of the combination is greater than the sum of the antimicrobial effects of the individual components).

In particular, non-limiting embodiments of the invention, the compositions comprise between about 0.1 and 1.0 percent (weight/weight)(“w/w”) of one or more essential oils, one or more ICs, or a combination thereof (where a combination is used, the total of essential oil(s) and/or IC(s) is between about 0.1 and 1.0 percent (weight/weight) and between about 0.125 and 2.0 percent (weight/weight) of one or more fruit acid (where more than one fruit acid is used, the total amount of fruit acids present is between about 0.125 and 2.0 percent (weight/weight)). “About” as used in this document means plus or minus 20 percent of the recited value, so that, for example, “between about 0.125 and 1.0 percent” means a range between 0.125±0.025 and 1.0±0.2.

In particular, non-limiting embodiments, the present invention provides for concentrates of essential oil/IC/fruit acid combinations which are concentrated and may be diluted to provide a composition for personal, household, or industrial use. In such concentrates, the ratio of fruit acid to essential oil(s)/IC(s) (weight/weight) is between about 1 and 16, for example, but not by way of limitation, fruit acid(s): EO(s)/IC(s) of between about 1:1 to 10:1, inclusive (weight/weight).

The present invention further provides for methods of providing an antimicrobial effect to a surface comprising applying, to the surface, an effective amount of a composition as described herein. An antimicrobial effect means killing and/or inhibiting the growth/proliferation of a microbe. In particular non-limiting embodiments of the invention, the microbe is selected from the group consisting of from the group consisting of Escherichia coli, Pseudomonas aeruginosa, Staphylococcus aureus, methicillin-resistant S. aureus, and Candida albicans. In specific non-limiting embodiments, the composition is exposed to the surface for at least 20 seconds, at least 30 seconds, or at least 60 seconds, or at least 5 minutes or at least 10 minutes. In various non-limiting embodiments, the surface may be the a skin or mucosal surface, a household surface (e.g., a surface of a countertop, table sink, toilet, wall, floor, appliance, window, shower surface, rug, upholstery, fabric, etc.) or an industrial surface (e.g., a surface of a countertop, table sink, toilet, wall, floor, appliance, window, shower surface, rug, upholstery, fabric, etc.).

In a first set of specific, non-limiting embodiments, the present invention provides for a composition comprising a component selected from the group consisting of cinnamon oil, cinnamaldehyde, eugenol, cinnamylacetic ester, and cinnamic acid, at a concentration of between about 0.1 and 1.2 percent (weight/weight) or between about 0.2 and 0.6 percent (weight/weight), as well as citric acid at a concentration of between about 0.5 and 1.5 percent (weight/weight), optionally further comprising triclosan at a concentration of between about 0.05 and 3 percent (weight/weight) or between about 0.05 and 0.1 percent (weight/weight) (this range, and all ranges herein, inclusive). In certain embodiments, the EO/IC is not cinnamon oil or pine oil or an IC thereof.

In a second set of non-limiting embodiments, the present invention provides for compositions comprising a EO/IC mixture comprising two or more EO or IC from the group consisting of cinnamon oil or an IC thereof, lemongrass oil and/or an IC thereof, orange oil and/or an IC thereof, basil oil and/or an IC thereof, and citronella oil and/or an IC thereof, at a total EO/IC concentration of between about 0.1 and 1 percent (weight/weight); together with one or more fruit acid (preferably citric acid), at a total fruit acid concentration of between about 0.125 and 2 percent (weight/weight); and an alcohol (preferably ethanol at a concentration of between about 5-20 percent (weight/weight), optionally further comprising triclosan at a concentration of between about 0.05 and 3 percent (weight/weight) or between about 0.05 and 0.1 percent (weight/weight), where the ratio of EO/IC to fruit acid is between about 1:1 to about 1:10. In certain embodiments, the EO/IC is not cinnamon oil or pine oil or an IC thereof.

In a third set of non-limiting embodiments, the present invention provides for compositions comprising a EO/IC mixture comprising lemongrass oil and/or an IC thereof, orange oil and/or an IC thereof, and optionally one or more additional EO and/or IC, at a total EO/IC concentration of between about 0.1 and 1 percent (weight/weight); together with one or more fruit acid (preferably citric acid), at a total fruit acid concentration of between about 0.125 and 2 percent; and an alcohol (preferably ethanol) at a concentration of between about 5-20 percent (weight/weight), optionally further comprising triclosan at a concentration of between about 0.05 and 1 percent (weight/weight) or between about 0.05 and 0.3 percent (weight/weight), where the ratio of EO/IC to fruit acid is between about 1:1 to about 1:10.

### 4.4 Personal Care Products

In non-limiting embodiments, the present invention provides for personal care product compositions comprising a combination of one or more essential oil and/or IC together with one or more fruit acid, as set forth in section 4.3, above. In preferred, non-limiting embodiments, the amounts of the active agents are such that regular exposure of skin to the personal care product does not produce skin irritation in a normal subject.

Non-limiting examples of personal care products which may utilize the invention include bar soap, liquid soap (e.g. hand soap), hand sanitizer, cleansing wipes, body wash, acne treatment products, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, body lotion, hand cream, topical cream, aftershave lotion, skin toner, mouth wash, toothpaste, sunscreen lotion, and baby products such as, but not limited to, cleansing wipes, baby shampoo, baby soap, and diaper cream.

Personal care compositions according to the invention, in addition to one or more essential oil and/or IC together with one or more fruit acid, may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, as well as additional components as may be known in the art. Non-limiting examples of such components are set forth below.

In various non-limiting embodiments of the invention, a personal care product comprising a combination of one or more essential oil and/or IC together with one or more fruit acid may further comprise an emollient, for example PEG 20 almond glycerides, Probutyl DB-10, Glucam P-20, Glucam E-10, Glucam P-10, Glucam E-20, Glucam P-20 distearate, glycerin, propylene glycol, octoxyglycerine, cetyl acetate, acetylated lanolin alcohol (e.g., Acetulan), cetyl ether (e.g., PPG-10), myristyril ether (e.g., PPG-3), hydroxylated milk glycerides (e.g., Cremeral HMG), polyquaternium compounds (e.g., U-care compounds), copolymers of dimethyl dialyl ammonium chloride and acrylic acid (e.g., Merquat), dipropylene glycol methyl ethers (e.g., Dowanol DPM, Dow Corning), polypropylene glycol ethers (e.g., Ucon 50-HB-600, Union Carbide) and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C₁₂-C₁₆ fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Additional useful emollients include lanolin, olive oil, cocoa butter, and shea butter.

In various non-limiting embodiments of the invention, a personal care product comprising a combination of one or more essential oil and/or IC together with one or more fruit acid may further comprise a stabilizing agent consisting of antioxidants, including but not limited to vitamin C (ascorbic acid) and vitamin E (tocopherol), and surfactants, including but not limited to incromide or silicone-based surfactants (Masil SF-19, BASF).

In various non-limiting embodiments of the invention, a personal care product comprising a combination of one or more essential oil and/or IC together with one or more fruit acid may further comprise a thickening agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof.

In various non-limiting embodiments of the invention, a personal care product comprising a combination of one or more essential oil and/or IC together with one or more fruit acid may further comprise a humectant, such as, for example, glycerin, 1-2-propylene glycol, dipropylene glycol, polyethylene glycol, 1,3-butylene glycol, or 1,2,6-hexanetriol.

In certain non-limiting embodiments of the invention, essentially the entire antimicrobial effect of the inventive composition is achieved by an antimicrobial composition consisting of one or more essential oil and/or one or more IC, together with a fruit acid and optionally an alcohol. In alternative embodiments of the invention, one or more additional antimicrobial agent may be comprised, for example, in the amount of between about 0.05 and 2.0 percent (weight/weight), where such antimicrobial agent may be selected from the group consisting of iodophors, iodine, benzoic acid, dihydroacetic acid, propionic acid, sorbic acid, methyl paraben, ethyl paraben, propyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, chlorhexidine embonate, and parahexamethylenebiguanide (“PHMB”).

In various non-limiting embodiments of the invention, a personal care product comprising a combination of one or more essential oil and/or IC together with one or more fruit acid may further comprise a neutralizing agent to neutralize carboxyl groups present in one or more other component, such as carboxyl groups in a thickening agent. Suitable neutralizing agents include diisopropylamine and triethanolamine.

In various non-limiting embodiments of the invention, a personal care product comprising a combination of one or more essential oil and/or IC together with one or more fruit acid may futher comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ”® nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. A preferred surfactant is lauroyl ethylenediamine triacetic acid sodium salt at a concentration between about 0.5-2.0%. In particular non-limiting embodiments of the invention, concentrations of surfactant are between about 0.05% and 2%.

In various non-limiting embodiments of the invention, a personal care product may comprise water.

In various non-limiting embodiments of the invention, a personal care product comprising a combination of one or more essential oil and/or IC together with one or more fruit acid may further comprise a hydrogel comprising, for example, a compound such as hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), and chitosan pyrrolidone carboxylate (Kytomer PC).

In various non-limiting embodiments of the invention, a personal care product comprising a combination of one or more essential oil and/or IC together with one or more fruit acid may further comprise an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol.

In various non-limiting embodiments of the invention, a personal care product comprising a combination of one or more essential oil and/or IC together with one or more fruit acid may further comprise a silicone polymer, for example one or more than one polydimethylsiloxane polymer (Dow Coming 225 Silicone Fluid), dimethiconol fluid in dimethicone (Dow Coming 1403 Silicone Fluid), cyclomethicone and dimethicone copolyl (Dow Coming 3225C Silicone Fluid), and silicone glycol (BASF 1066 DCG polyol). In particular, non-limiting embodiments, the amount of silicone polymer is between about 0.1 and 1.0 percent (volume/volume).

In various non-limiting embodiments of the invention, a personal care product comprising a combination of one or more essential oil and/or IC together with one or more fruit acid may further comprise an emollient solvent such as a glycidyl ether having an alkyl chain up to and including 18 carbon molecules and ethoxylates and propoxylates thereof, a glyceryl ether having an alkyl chain up to and including 18 carbon molecules and ethoxylates and propoxylates thereof, a mono- or diglyceryl ether having an alkyl chain up to and including 18 carbon molecules and ethoxylates and propoxylates thereof, ethoxylate and propoxylate ethers, ethoxy diglycol esters, ethyl hexyl alcohol propoxylate, and propylene glycol esther ethoxylates and propoxylates, and Arlamol (Altas).

In various non-limiting embodiments of the invention, a personal care product comprising a combination of one or more essential oil and/or IC together with one or more fruit acid may futher comprise additives such as dyes, fragrances, pH adjusters, including basic pH adjusters such as ammonia, mono-, di- and tri- alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

In one set of non-limiting embodiments, the present invention provides for personal care compositions comprising one or more EO/IC, preferably where the EO(s)/IC(s) are selected from the group consisting of lemongrass oil and/or an IC thereof, orange oil and/or an IC thereof, cinnamon leaf oil and/or an IC thereof, basil oil and/or an IC thereof, eugenol, cinnamaldehyde, cinnamylacetic ester, and cinnamic acid, at a total concentration of between about 0.1 and 1 percent (weight/weight); a fruit acid, preferably citric acid, at a concentration of between about 0.125 and 1 percent (weight/weight); an alcohol, preferably ethanol, at a concentration of between about 5 and 20 percent (weight/weight); and optionally triclosan at a concentration of between about 0.05 and 1 percent (weight/weight), where the ratio of EO(s)/IC(s) to the fruit acid(s) is between about 1:1 to 1:10 and the pH is between about 3 and about 7, preferably between 5 and 6.

In another set of non-limiting embodiments, the present invention provides for personal care compositions comprising lemograss oil or an IC thereof and orange oil or an IC thereof at a total concentration of between about 0.2 and 0.7 percent (weight/weight); a fruit acid, preferably citric acid, at a concentration of between about 0.25 and 1 percent (weight/weight); an alcohol, preferably ethanol, at a concentration of between about 5 and 20 percent (weight/weight); and optionally triclosan at a concentration of between about 0.05 and 1 percent (weight/weight), where the ratio of EO(s)/IC(s) to fruit acid(s) is between about 1:1 to 1:5 and the pH is between about 3 and about 7, preferably between 5 and 6.

In one specific, non-limiting embodiment, the present invention provides for a liquid soap product called “CN1-A” having the following composition:

| | | |
|---|---|---|
| Deionized water | 59.15% | (weight/weight) |
| Polyox N 60K | 0.2% | |
| Pluronic F 87 Prill | 2.0% | |
| Ucare Jr 30 | 0.4% | |
| D,L Panthenol 50 W | 1.0% | |
| Incromide oxide L | 3.0% | |
| Crosultane C-50 | 3.0% | |
| Montalene C 40 | 3.0% | |
| 2-Phenoxy-ethanol | 1.0% | |
| Zinc gluconate | 0.1% | |
| Glycerine | 2.0% | |
| SDA-40B alcohol | 15.5% | |
| Cinnamon leaf oil | 0.5% | |
| Citric acid | 1.0% | |
| Orange oil | 0.2% | |
| Distilled water | 7.95% | |

In another specific, non-limiting embodiment, the present invention provides for a liquid soap product called “CN1-B” having the following composition:

| | | |
|---|---|---|
| Deionized water | 63.2% | (weight/weight) |
| Methocel 40-101 | 0.1% | |
| Pluronic F 87 Prill | 0.1% | |
| Ucare Jr 30 | 0.1% | |
| D,L Panthenol 50 W | 1.0% | |
| Incromide oxide L | 3.0% | |
| Crosultane C-50 | 3.0% | |
| Montalene C 40 | 1.5% | |
| 2-Phenoxy-ethanol | 1.0% | |
| Glycerine | 2.0% | |
| SDA-40B alcohol | 15.5% | |
| Cinnamon leaf oil | 0.5% | |
| Citric acid | 1.0% | |
| Orange oil | 0.2% | |
| Distilled water | 7.8% | |

In another specific, non-limiting embodiment, the present invention provides for a liquid soap product called “CN1-C” having the following composition:

| | | |
|---|---|---|
| Deionized water | 63.2% | (weight/weight) |
| Methocel 40-101 | 0.1% | |
| Pluronic F 87 Prill | 0.1% | |
| Ucare Jr 30 | 0.1% | |
| D,L Panthenol 50 W | 1.0% | |
| Incromide oxide L | 3.0% | |
| Crosultane C-50 | 3.0% | |
| Montalene C 40 | 1.5% | |
| 2-Phenyl-ethanol | 1.0% | |
| Glycerine | 2.0% | |
| SDA-40B alcohol | 15.5% | |
| Cinnamon leaf oil | 0.5% | |
| Citric acid | 1.0% | |
| Orange oil | 0.2% | |
| Distilled water | 7.8% | |

In a subset of non-limiting embodiments, the present invention provides for a soap comprising one or more essential oil, 1% citric acid, and a soap base comprising a surfactant, an emollient, and a thickener, and having a pH between about 3-5. Specific non-limiting examples of such soaps follow.

Soap Containing Lemongrass oil, and Citric acid (LG-Cit-4) (4 represents total oil 0.4%)

| Ingredient | Percentage (w/w) |
|---|---|
| Deionized Water | 63.5 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.4 |

Soap Containing Lemongrass oil, and Citric acid (LG-Cit-6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.6 |

Soap Containing Lemongrass oil, Orange oil(O oil) and Citric acid (LGO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.4 |
| Citric acid | 1.0 |
| Orange oil | 0.2 |

Soap Containing Lemon grass oil , Orange oil and Citric acid (LGO-Cit 7) (7 represents total oil 0.7%)

| Ingredient | Percentage (w/w) |
|---|---|
| Deionized Water | 63.2 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.5 |
| Citric acid | 1.0 |
| Orange oil | 0.2 |

Soap Containing Cinnamon oil, Orange oil and Citric acid (CO -Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| DL Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Cinnamon oil | 0.4 |
| Citric acid | 1.0 |
| Orange oil | 0.2 |

Soap Containing Cinnamon oil, Orange oil and Citric acid (CO-Cit 7) (7 represent total oil 0.7%)

| Ingredient | Percentage (w/w) |
|---|---|
| Deionized Water | 63.2 |
| Methocel 40-101 | 0.1 |
| Pluronic F 87 Prill | 0.1. |
| Ucare Jr 30 | 0.1 |
| D, L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Cinnamon oil | 0.5 |
| Citric acid | 1.0 |
| Orange oil | 0.2 |

Soap Containing Orange oil and Citric acid (O-Cit 2) (2 represents total oil 0.2%)

| Ingredient | Percentage (w/w) |
|---|---|
| Deionized Water | 63.7 |
| Methocel 40-101 | 0.1 |
| Pluronic F 87 Prill | 0.1. |
| Ucare Jr 30 | 0.1 |
| D, L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Orange oil | 0.2 |
| Citric acid | 1.0 |

Soap Containing Basil oil (“B oil”), Orange oil(“O oil”) and Citric acid (BO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D, L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Basil oil | 0.4 |
| Citric acid | 1.0 |
| Orange oil | 0.2 |

Soap Containing Citronella oil (“CR oil”), Orange oil(“O oil”) and Citric acid (CRO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D, L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Citronella oil | 0.4 |
| Citric acid | 1.0 |
| Orange oil | 0.2 |

In further specific, non-limiting embodiments, the present invention provides for the following combinations of agents in a soap base (percentages w/w):

0.15% TC+0.4% lemongrass oil+0.2% orange oil+1% citric acid;

0.4% lemongrass oil+0.2% orange oil+1% citric acid; or

0.15% TC+0.4% cinnamon oil+0.2% orange oil+1% citric acid; or

0.4% cinnamon oil+0.2% orange oil+1% citric acid.

In still further specific, non-limiting embodiments, the present invention provides for the following combinations of agents in a soap base (percentages w/w):

Cinnamon oil 0.5%+OrangeOil 0.2%+Citric acid 1.0%+alcohol (e.g., denatured ethyl alcohol, such as SDA 40 B) 5.5%+TC 0.14% (or TC 0.15%); or

Lemongrass oil 0.5%+Orange Oil 0.2%+Citric acid 1.0%+alcohol (e.g., denatured ethyl alcohol, such as SDA 40B) 5.5%+TC 0.14% (or TC 0.15%); or

Lemongrasss oil 0.5%+Citric acid 1.0%+alcohol (e.g. denatured ethyl alcohol such as SDA 40 B)5.5%+TC 0.14% (or TC 0.15%).

### 4.5 Veterinary Products

In a subset of non-limiting embodiments, the present invention provides for veterinary products comprising a combination of one or more essential oil and/or IC together with one or more fruit acid, as set forth in section 4.3, above. The term “veterinary”, as used here, means “pet care”, and includes home use as well as use in a veterinary office or other pet care establishment.

Non-limiting examples of veterinary care products which may utilize the invention include pet shampoo, pet cleansing wipes including body wipes, ear wipes, and eye wipes, ear cleaning liquid, cage cleaner, surface cleaner for housebreaking accidents, topical creams, teat dip therapeutic for mastitis and liquid to be applied to pet's skin (as in a “body splash”).

Veterinary care compositions according to the invention, in addition to one or more essential oil and/or IC together with one or more fruit acid, may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, anti-inflammatory agents, wound healing agents, as well as additional components as may be known in the art.

Specific, non-limiting examples of additional components which may be comprised in pet care products include the components listed above for personal care products.

### 4.6 Household/Industrial Products

In a subset of non-limiting embodiments, the present invention provides for household/industrial products comprising a combination of one or more essential oil and/or IC together with one or more fruit acid, as set forth in section 4.3, above.

Non-limiting embodiments of household/industrial products which may utilize the invention include householder cleaners such as concentrated liquid cleaners and spray cleaners, cleaning wipes, dish washing liquid, dish washer detergent, spray-mop liquid, furniture polish, indoor paint, outdoor paint, dusting spray, laundry detergent, fabric softener, rug/fabric cleaner, window and glass cleaner, toilet bowl cleaner, liquid/cream cleanser, etc. In a particular embodiment, the invention may be used in a food wash product, designed to clean fruits and vegetables prior to consumption. “Household products” are products, other than personal care products, that would be used by individual consumers. “Industrial products” refers to products that are used in industry.

Household-industrial compositions according to the invention, in addition to one or more essential oil and/or IC together with one or more fruit acid, may further comprise one or (preferably) more than one component selected from the group consisting of surfactants, builders (e.g., sequestering builders, precipitating builders, ion exchange builders), solvents, thickeners, abrasives, acids, bases (alkalis), antimicrobial agents, soaps, bleaching agents, enzymes, preservatives, and sudsing agents, as well as additional components as may be known in the art.

In various non-limiting embodiments of the invention, a household/industrial product comprising a combination of one or more essential oil and/or IC together with one or more fruit acid may further comprise a surfactant, for example, but not limited to, an anionic surfactant such as an alkyl sulfate, an alkyldiphenyloxide disulfonate salt ( e.g., the DOWFAX series by the Dow Chemical Company), an alkylbenzenesulfonate, an alcohol ethoxysulfate; a cationic surfactant; a non-ionic surfactant, such as a secondary alcohol ethoxylate (e.g., the TERGITAOL series by the Dow Chemical Company) or an alkyl polyglucoside (e.g. the TRITON series by the Dow Chemical Company); or an amphoteric surfactant such as an imidazoline or betaine compound.

In various non-limiting embodiments of the invention, a household/industrial product comprising a combination of one or more essential oil and/or IC together with one or more fruit acid may further comprise a builder, for example, but not limited to, a sequestering builder (chelating agent) such as ethylenediaminetetraacetic acid (“EDTA”), sodium citrate, or a complex phosphate; an ion exchange builder such as zeolite, or a precipitating builder such as sodium carbonate or sodium silicate.

In various non-limiting embodiments of the invention, a household/industrial product comprising a combination of one or more essential oil and/or IC together with one or more fruit acid may further comprise a solvent, for example, but not limited to, water, an alcohol such as methanol, ethanol, isopropyl alcohol, or butanol; a hydrocarbon such as an aromatic hydrocarbon, prolylene glycol, methylene chloride, acetone, a petroleum distillate, and/or a glycol ether.

In various non-limiting embodiments of the invention, a household/industrial product comprising a combination of one or more essential oil and/or IC together with one or more fruit acid may further comprise a thickener, for example, but not limited to, a polyethylene glycol a methoxypolyethylene glycol, and/or hydroxyethyl cellulose.

In various non-limiting embodiments of the invention, a household/industrial product comprising a combination of one or more essential oil and/or IC together with one or more fruit acid may further comprise an abrasive, such as, but not limited to, silica, feldspar or calcite.

In various non-limiting embodiments of the invention, a household/industrial product comprising a combination of one or more essential oil and/or IC together with one or more fruit acid may further comprise an acid, such as, but not limited to, acetic acid, hydroacetic acid, phosphoric acid or hydrochloric acid.

In various non-limiting embodiments of the invention, a household/industrial product comprising a combination of one or more essential oil and/or IC together with one or more fruit acid may further comprise a base (alkali) such as, but not limited to, ammonia or sodium bicarbonate.

In various non-limiting embodiments of the invention, a household/industrial product comprising a combination of one or more essential oil and/or IC together with one or more fruit acid may further comprise an antimicrobial agent, for example, but not limited to, compounds as set forth above for personal care compositions, and also pine oil and sodium hypochlorite.

In various non-limiting embodiments of the invention, a household/industrial product comprising a combination of one or more essential oil and/or IC together with one or more fruit acid may further comprise a bleaching agent, for example, but not limited to, sodium hypochlorite, hydrogen peroxide, sodium percarbonate and sodium perborate.

In various non-limiting embodiments of the invention, a household/industrial product comprising a combination of one or more essential oil and/or IC together with one or more fruit acid may further comprise an enzyme, such as, but not limited to, a protease or a lipase.

In various non-limiting embodiments of the invention, a household/industrial product comprising a combination of one or more essential oil and/or IC together with one or more fruit acid may further comprise a preservative, such as, but not limited to, butylated hydroxytoluene, glutaraldehyde, and EDTA.

In various non-limiting embodiments of the invention, a household/industrial product comprising a combination of one or more essential oil and/or IC together with one or more fruit acid may further comprise a sudsing agent, such as, but not limited to, diethanolamine or triethanolamine.

In one set of non-limiting embodiments, the present invention provides for surface cleaner compositions comprising (i) one or more EO/IC, preferably where the EO(s)/IC(s) are selected from the group consisting of lemongrass oil and/or an IC thereof; orange oil and/or an IC thereof; cinnamon leaf oil and/or an IC thereof; basil oil and/or an IC thereof; and/or pine oil and/or an IC thereof; at a total concentration of between about 0.1 and 1 percent (weight/weight); (ii) a fruit acid, preferably citric acid, at a concentration of between about 1 and 2 percent (weight/weight); (iii) an alcohol, preferably ethanol, at a concentration of between about 5 and 20 percent (weight/weight); and (iv) optionally triclosan at a concentration of between about 0.05 and 1 percent (weight/weight), where the ratio of EO(s)/IC(s) to fruit acid is between about 1:1 to 1:10 (inclusive) and the pH is between about 3 and about 7, preferably between 3 and 5. In certain non-limiting embodiments of the invention, cinnamon leaf oil or an IC thereof and/or pine oil or an IC thereof is not present.

In specific, non-limiting embodiments, the present invention provides for the following surface cleaners, having concentrations of active ingredients as indicated, as well as concentrated stock solutions of these formulations which may be diluted to achieve the respective concentrations:

Surface Disinfectant-LG cit 2
0.2% Lemon grass oil+2% Citric acid+7.65% Alcohol+0.15% surfactants

Surface Disinfectant-LG P cit 4
0.3% Pine oil+0.1% Lemon grass oil+2% Citric Acid+7.45% alcohol+0.15% Surfactants

Surface Disinfectant-P cit 5
0.5% Pine oil+2% Citric acid+7.45% alcohol+0.15% surfactants.

Surface Disinfectant-PO Cit 7
0.5% Pine oil+0.2% Orange oil+1% Citric Acid+5.35% alcohol+0.15% Surfactants

Surface Disinfectant-LGO Cit 7
0.5% lemongrass oil+0.2% Orange oil+1% Citric Acid+5.35% alcohol+0.15% Surfactants

The detailed description hereby incorporates, by reference, the specific working examples of the invention set forth below.

The working examples sometimes refer to Softsoap® or Dial® soaps.

Softsoap® is a commercially sold liquid soap comprising water, sodium laureth sulfate, cocamidopropyl betaine, decylglucoside, sodium chloride, fragrance, DMDM hydantoin, PEG-120 methyl glucose dioleate, tetrasodium ethylene diamine tetracetic acid, sodium sulfate, polyquaternium-7, citric acid, poloxamer 124, PEG-7 glyceryl, cocoate, benzophenine-4, and colors.

Dial® soap is a commercially sold liquid soap, where Dial® Antibacterial hand coap comprises, as active agent, 0.15 percent triclosan, and the inactive agents are water, sodium laureth sulfate, ammonium lauryl sulfate, decyl glucoside, cocamidopropyl betaine, glycerine, sodium chloride, PEG-18 gylceryl oleate/cocoate, fragrance, cocamide MEA, DMDM hydantoin, tetrasodium ethylene diamine tetracetic acid and colors.

### 5. EXAMPLE 1

Various concentrations of basil oil and acetic, lactic, and citric acids, separately and in combination, were prepared in 10 percent SDA40-B alcohol and water, and adjusted to 100 percent. Except for citric acid, which was added by weight, all other ingredients were measured by volume. 0.9 ml of each solution were dispensed in sterile culture tubes, in triplicate, and 0.1 ml of a 10⁷ cfu/ml S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 1. The greater synergy was observed between basil oil and citric acid (“CA”).

TABLE 1

| Compounds | Log 10 Reduction* |
|---|---|
| 1% Basil oil | 2.7 |
| 0.5% Basil oil | 1.8 |
| 0.25% Basil oil | 0.4 |
| 1% Acetic acid (AA) | 0.2 |
| 1% Lactic acid (LA) | 2.3 |
| 1% Citric Acid (CA) | 0.1 |
| 1% Basil oil + 1% LA | 5.4 |
| 1% Basil oil + 1% AA | 3.4 |
| 1% Basil oil + 1% CA | 5.1 |
| 0.5% Basil oil + 1% CA | 5.1 |
| 0.5% Basil oil + 0.5% CA | 5.0 |
| 0.25% Basil oil + 1% CA | 5.0 |
| 0.25% Basil oil + 0.5% CA | 2.5 |

*Log reduction from control bacterial counts ranging from 1 × 10⁶ to 5 × 10⁶.

The same methodology was used to test the antimicrobial activity of combinations of citric acid with other essential oils. The results are shown in Table 2. In these experiments, cinnamon oil and citronella oil exhibited superior antimicrobial activities in combination with citric acid.

TABLE 2

| Compounds | Log 10 Reduction* |
|---|---|
| 1% CA | 0.1 |
| 0.5% Cinnamon bark | 2.4 |
| 0.25% Cinnamon bark | 1.1 |
| 0.5% Cinnamon bark + 1% CA | 5.9 |
| 0.25% Cinnamon bark + 1% CA | 4.3 |
| 0.125% Cinnamon bark + 1% CA | 2.6 |
| 0.25% Cinnamon leaf | 2.8 |
| 0.25% Cinnamon leaf + 1% CA | 5.7 |
| 0.125% Citronella oil | 1.3 |
| 0.25% Citronella oil + 1% CA | 6.2 |
| 0.125% Citronella oil + 1% CA | 3.3 |
| 0.25% Orange oil | 0 |
| 0.25% Orange oil + 1% CA | 2.3 |
| 0.25% Lemon oil | 0.05 |
| 0.25% Lemon oil + 1% CA | 3.3 |
| 0.25% Lavender oil | 0.25 |
| 0.25% Lavender oil + 1% CA | 4.0 |
| 0.25% Clove oil | 0.1 |
| 0.25% Clove oil + 1% CA | 3.3 |
| 0.25% Tea tree oil | 0 |
| 0.25% Tea tree + 1% CA | 4.7 |
| 0.25% Farnesol | 0 |
| 0.25% Farnesol + 1% CA | 4.0 |

*Log reduction from control bacterial counts ranging from 1 × 10⁶ to 5 × 10⁶.

Next, the same general protocol was used to test the efficacy of basil, cinammon and citronella oils against a variety of organisms, namely E. col, P. aeruginosa, MRSA, C. albicans, and S. aureus. The results, which demonstrates that in these experiments, combinations of cinnamon oil and citric acid exhibited superior antimicrobial action, are shown in Table 3.

TABLE 3
Log 10 Reductions*

| | E. | P. | | C. | S. |
|---|---|---|---|---|---|
| Compounds | coli | aeruginosa | MRSA | albicans | aureus |
| 0.25% Basil oil + | 6.0 | 5.5 | 5.2 | 1.5 | 5.0 |
| 1% CA | | | | | |
| 0.25% Cinnamon bark | 6.0 | 6.0 | 5.2 | 4.5 | 4.3 |
| oil + 1% CA | | | | | |
| 0.25% Citronella oil + | 6.4 | 6.4 | 6.5 | 1.0 | 6.3 |
| 1% CA | | | | | |
| 0.25% Cinnamon leaf | 6.4 | 6.4 | 6.5 | 5.4 | 6.3 |
| oil + 1% CA | | | | | |
| 0.25% Eugenol + | — | 6.5 | — | 5.5 | — |
| 1% CA | | | | | |

*Log reduction from control bacteria counts ranging from 1 × 10⁶ to 5 × 10⁶ or C. albicans ranging from 1 × 10⁵ to 5 × 10⁵.

### 6. EXAMPLE 2

The following experiments were performed to evaluate the effectiveness of a hard surface cleaner composition comprising cinnamon leaf oil and citric acid.

Two stock solutions of a hard surface cleaner/disinfectant was prepared, with the following ingredients (the two solutions contained different amounts of cinnamon leaf oil, and therefore the amount of alcohol to bring the solution to 100% also varied):

| | |
|---|---|
| Cinnamon leaf oil | 3.6 or 7.2% (weight/weight (“w/w”)) |
| Citric acid | 14.3% (weight/weight) |
| SDA 40B alcohol | 77.2 or 75.49 (w/w) (to bring the |
| | volume to 100%) |
| Pluronic surfactant L-61 | 0.7% (w/w) |
| Pluronic surfactant F-127 | 0.7% (w/w) |
| Pluronic surfactant F-87 | 0.7% (w/w) |
| Orange oil | 2.8% (w/w) |

7% of the stock hard disinfectant was diluted with water to 100%.

0.1 ml of culture containing approximately 1×10⁷ colony forming units (“cfu”) per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀. Commercially available Pinesol®, which contains pine oil, was used as a basis for comparison. Pinesol® containing 15% pine oil was diluted with water as per the manufacturer's instructions to a final concentration of 0.9% pine oil. The results are shown in Table 4. The results show that the composition comprising 0.5% cinnamon leaf oil and 1% citric acid exhibited greater antimicrobial activity than the pine oil cleaner against 4 out of 5 microbes tested.

TABLE 4

| | Log 10 Reductions* | | |
|---|---|---|---|
| | 0.25% cinn. oil + | 0.5% cinn. oil + | |
| Organism | 1% CA | 1% CA | 0.1% Pinesol ® |
| E. coli | 5.3 | 5.3 | 5.7 |
| P. aeruginosa | 6.1 | 6.1 | 3.9 |
| MRSA | 2.3 | 3.4 | 2.2 |
| C. albicans | 2.5 | 5.2 | 2.1 |
| S. aureus | 3.7 | 4.1 | 2.4 |

*log₁₀ reduction from control bacterial counts (ranges from 1 × 10⁶-5 × 10⁶ for all bacteria, but for C. albicans counts were 1 × 10⁵-5 × 10⁵.

### 7. EXAMPLE 3

Various concentrations of cinnamon leaf oil and citric acid were dissolved in SDA 40-B alcohol (10%) and water, and adjusted to 100 percent. Except for citric acid, which was added by weight, all other ingredients were measured by volume. 0.9 ml of each solution were dispensed in sterile culture tubes, in triplicate, and 0.1 ml of 10⁷ cfu/ml of S. aureus culture was added to the tubes, vortexed, and then, five minutes later, 9.0 ml of drug inactivating medium was added to each tube. Serial dilutions were made with the drug inactivating medium. 0.5 ml of the dilutions were plated on trypticase soy agar (“TSA”) plates. As a control, water containing 10% percent SDA40-B alcohol was processed in parallel. The plates were incubated at 37° C. for 24-48 hours and then the colony counts were determined. The results are shown in Table 5.

TABLE 5

| Compounds | Log 10 reduction |
|---|---|
| Citric Acid 2% | 0.32 |
| Citric Acid 1.0% | 0.30 |
| Citric Acid 0.5% | 0.20 |
| Citric Acid 0.25% | 0.08 |
| Citric Acid 0.125% | 0.02 |
| Cinnamon leaf oil 0.25% | 0.52 |
| Cinnamon leaf oil 0.5% | 0.55 |
| 0.25% Cinnamon + 0.25% CA | 0.73 |
| 0.25% Cinnamon + 0.5% CA | 3.0 |
| 0.25% Cinnamon + 1.0% CA | 5.6 |
| 0.5% Cinnamon + 0.125% CA | 0.84 |
| 0.5% Cinnamon + 0.25% CA | 2.2 |
| 0.5% Cinnamon + 0.5% CA | 3.2 |
| 0.5% Cinnamon + 1.0% CA | 6.5 |
| 0.5% Cinnamon + 2.0% CA | 6.7 |

### 8. EXAMPLE 4

A liquid soap, called “CN1-A” containing cinnamon oil and citric acid was prepared, having the following composition:

| | | |
|---|---|---|
| Deionized water | 59.15% | (weight/weight) |
| Polyox N 60K | 0.2% | |
| Pluronic F 87 Prill | 2.0% | |
| Ucare Jr 30 | 0.4% | |
| D, L Panthenol 50 W | 1.0% | |
| Incromide oxide L | 3.0% | |
| Crosultane C-50 | 3.0% | |
| Montalene C 40 | 3.0% | |
| 2-Phenoxy-ethanol | 1.0% | |
| Glycerine | 2.0% | |
| SDA-40B alcohol | 15.5% | |
| Cinnamon leaf oil | 0.5% | |
| Citric acid | 1.0% | |
| Orange oil | 0.2% | |
| Distilled water | 7.95% | |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Softsoap® containing triclosan (Softsoap® Antibacterial; Colgate-Palmolive). 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 6.

TABLE 6

Log₁₀ reduction from control*

| Organisms | CN-1A | Softsoap ® (0.15% TC) |
|---|---|---|
| S. aureus | 2.0 | 0.33 |
| P. aeruginosa | 2.5 | 0.6 |
| E. coli | 4.86 | 0.5 |
| MRSA | 2.7 | 0.8 |
| C. albicans | 1.43 | 0.0 |

*log₁₀ reduction from control microbe counts which in all cases ranged from 1 × 10⁷-5 × 10⁷.

### 9. EXAMPLE 5

A liquid soap, called “CN1-B” containing cinnamon oil and citric acid was prepared, having the following composition:

| | | |
|---|---|---|
| Deionized water | 63.2% | (weight/weight) |
| Methocel 40-101 | 0.1% | |
| Pluronic F 87 Prill | 0.1% | |
| Ucare Jr 30 | 0.1% | |
| D, L Panthenol 50 W | 1.0% | |
| Incromide oxide L | 3.0% | |
| Crosultane C-50 | 3.0% | |
| Montalene C 40 | 1.5% | |
| 2-Phenoxy-ethanol | 1.0% | |
| Glycerine | 2.0% | |
| SDA-40B alcohol | 15.5% | |
| Cinnamon leaf oil | 0.5% | |
| Citric acid | 1.0% | |
| Orange oil | 0.2% | |
| Distilled water | 7.8% | |

To prepare the soap, cinnamon oil orange oil, citric acid, and phenoxyethanol are dissolved in the alcohol, the remaining ingredients are dissolved in/mixed with water, and then the alcohol and water solutions are mixed. The pH of the mixture was then adjusted to between 5.5 and 6.5 with 0.1 N NaOH.

The antimicrobial activity of the above soap was tested in parallel with commercial Dial® Antibacterial Hand Soap) containing triclosan. 0.1 ml of a 10⁸ cfu/ml culture of each microbe tested was mixed with 0.1 ml of bovine serum and placed in a sterile culture tube. 0.8 ml of the test soap formulation was added to the tube and vortexed for 30 seconds. Then 9.0 ml DNB was added to neutralize the activity of the soap. The tube was then vortexed and serially diluted with DNB. 0.5 ml of the diluted solution was plated on TSA plates. The same soap base lacking cinnamon oil, citric acid, and orange oil, with phosphate buffered saline mixed with the culture, were used as the controls. The results are shown in Table 7.

TABLE 7

Log₁₀ reduction from control*

| | | Dial ® soap (0.15% |
|---|---|---|
| Organisms | CN1-B | TC) |
| S. aureus | 5.0 | 0.36 |
| MRSA | 5.1 | 0.03 |
| E. coli | 4.45 | 0 |
| P aeruginosa | 5.9 | 0.12 |

*log₁₀ reduction from control microbe counts which in all cases ranged from 1 × 10⁷-5 × 10⁷ 3.4 × 10⁶ for S. aureus, 3-5 × 10⁶ for E. coli and 6 × 10⁵-1.3 × 10⁶ for MRSA.

### 10. EXAMPLE 6

The effectiveness of Softsoap® Juicy Melon (Colgate-Palmolive) with added cinnamon oil, citric acid, and/or triclosan, against MRSA was evaluated. Testing was performed essentially as set forth in the preceding section 9. The results are shown in Table 8.

TABLE 8

| Compounds | Log 10 reduction* |
|---|---|
| Soft Soap ® + 1.5% cin-cit | 3.63 |
| Soft soap ® + 0.075% TC | 0.15 |
| Soft soap ® + 0.15% TC | 0.20 |
| Soft soap ® + 0.3% TC | 0.58 |
| Soft soap ® + 0.075% TC + 1.5% Cin- | 4.29 |
| Cit | |
| Soft soap ® + 0.15% TC + 1.5% Cin-Cit | 4.87 |
| Soft soap ® + 0.3% TC + 1.5% Cin-Cit | 6.38 |

*log₁₀ reduction from control microbe counts which in all cases ranged from 1 × 10⁶-5 × 10⁶.

### 11. EXAMPLE 7

The ability of cinnamon oil and citric acid to potentiate the activity of commercial triclosan-containing soaps such as Softsoap® and Dial® Antibacterial Hand Soap containing 0.15% triclosan was tested using an assay essentially as set forth in Section 9, above. The results are shown in Table 9.

TABLE 9

| | Log₁₀ reduction from control* | | |
|---|---|---|---|
| | S. aureus | E. Coli | MRSA |
| Soft Soap ®-TC | 0.33 | 0.25 | 0.37 |
| Soft Soap ®-TC + CIN- | 3.9 | 3.93 | 6.0 |
| Cit | | | |
| Dial ® Soap-TC | 0.36 | 0 | 0.24 |
| Dial ® Soap-TC + Cin-Cit | 3.74 | 4.18 | 6.0 |

*Log reduction from control bacterial counts (ranges from 3 4 × 10⁶ for S. aureus, 3-5 × 10⁶ for E. coli and 6 × 10⁵-1.3 × 10⁶ for MRSA.

In these experiments, the combination of cinnamon oil and citric acid was found to substantially improve the antimicrobial activity of the commercial soap.

### 12. EXAMPLE 8

Because a major ingredient of cinnamon oil is eugenol, the effect of adding eugenol on the antimicrobial activity of commercial soaps was also tested. The assay was essentially as set forth in Section 9, above. The results are shown in Table 10.

TABLE 10

| | Log reduction from control* |
|---|---|
| | S. aureus |
| Dial ® Soap-TC | 0.30 |
| Dial ® Soap-TC + 0.5% | 2.32 |
| Eugenol + 1% CA | |
| Dial ® Soap-TC + 0. | 3.94 |
| 0.5% cinnamon oil + 1% CA | |

*Log reduction from control bacterial counts (ranged from 3-4 × 10⁶ for S. aureus).

These experiments showed that while adding eugenol improved the antimicrobial effect, the improvement was not as great as that observed for cinnamon oil.

### 13. EXAMPLE 9

The following experiments were performed to evaluate the antibacterial activity of LG and Citric acid dissolved in alcohol, where the test organism used was S. aureus. Various amounts of LG oil and Citric acid were dissolved in SDA40-B alcohol, and then water was added to result in the EO concentration shown and an alcohol concentration of 10 percent. 0.9 ml of each solution were dispensed in sterile culture tubes, in triplicate, and 0.1 ml of a 10⁷ cfu/ml S. aureus culture was added to the tubes, vortexed, and then, five minutes later, 9.0 ml of drug inactivating medium was added to each tube. Serial dilutions were made with drug inactivating medium. 0.5 ml of the dilutions were plated on trypticase soy agar (“TSA”) plates. As a control, water containing 10 percent SDA40-B alcohol was processed in parallel. The plates were incubated at 37° C. for 24-48 hours and then the colony counts were determined. The results are shown in Table 11.

TABLE 11

| Compounds | Log 10 reduction from control |
|---|---|
| 1% Citric acid | 0.3 |
| 0.5% LG oil | 1.24 |
| 0.55 LG oil + 1% Citric acid | 5.59 |

*Log 10 reduction from control bacterial counts (control counts ranges from 1 × 10⁶ to 5 × 10⁶)

The results shown in Table 11 indicate that LG oil exhibits superior anti bacterial action in combination with Citric acid.

### 14. EXAMPLE 10

Soaps were prepared containing one or more essential oil, 1% citric acid, and a soap base containing surfactants, emollients, thickeners etc. The pH of the Soaps ranged from 3.2-3.3.

Soap Containing Lemongrass oil, and Citric acid (LG-Cit-4) (4 represents total oil 0.4%)

| Ingredient | Percentage (w/w) |
|---|---|
| Deionized Water | 63.5 |
| Methocel 40-101 | 0.1 |
| Pluronic F 87 Prill | 0.1 |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.4 |

Soap Containing Lemongrass oil, and Citric acid (LG-Cit-6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.6 |

Soap Containing Lemongrass oil, Orange oil(O oil) and Citric acid (LGO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.4 |
| Citric acid | 1.0 |
| Orange oil | 0.2 |

Soap Containing Lemon grass oil, Orange oil and Citric acid (LGO-Cit 7) (7 represents total oil 0.7%)

| Ingredient | Percentage (w/w) |
|---|---|
| Deionized Water | 63.2 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Lemongrass oil | 0.5 |
| Citric acid | 1.0 |
| Orange oil | 0.2 |

Soap Containing Cinnamon oil, Orange oil and Citric acid (CO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| DL Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Cinnamon oil | 0.4 |
| Citric acid | 1.0 |
| Orange oil | 0.2 |

Soap Containing Cinnamon oil, Orange oil and Citric acid (CO -Cit 7) (7 represent total oil 0.7%)

| Ingredient | Percentage (w/w) |
|---|---|
| Deionized Water | 63.2 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Cinnamon oil | 0.5 |
| Citric acid | 1.0 |
| Orange oil | 0.2 |

Soap Containing Orange oil and Citric acid (O-Cit 2) (2 represents total oil 0.2%)

| Ingredient | Percentage (w/w) |
|---|---|
| Deionized Water | 63.7 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Orange oil | 0.2 |
| Citric acid | 1.0 |

Soap Containing Basil oil (“B oil”), Orange oil(“O oil”) and Citric acid (BO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Basil oil | 0.4 |
| Citric acid | 1.0 |
| Orange oil | 0.2 |

Soap Containing Citronella oil (“CR oil”), Orange oil(“O oil”) and Citric acid (CRO-Cit 6) (6 represents total oil 0.6%)

| Ingredient | Percentage (w/w) |
|---|---|
| Deionized Water | 63.3 |
| Methocel 40-101 | 0.1 |
| Pluronic F 87 Prill | 0.1. |
| Ucare Jr 30 | 0.1 |
| D,L Panthenol 50 W | 1.0 |
| Incromide Oxide L | 3.0 |
| Crosultane C-50 | 3.0 |
| Montalene C 40 | 1.5 |
| 2-Phenoxy-Ethanol | 1.0 |
| Glycerin | 2.0. |
| SDA 40B | 15.5 |
| Citronella oil | 0.4 |
| Citric acid | 1.0 |
| Orange oil | 0.2 |

### 15. EXAMPLE 11

Certain soaps prepared in Example 14 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. Soft soap® and Dial® soaps containing 0.15% triclosan was also tested similarly at the same time. The soap base without essential oils and Citric acid containing the culture were used as controls. The results, showing 30 second kill activity, are shown in Table 12.

TABLE 12

| Soap | Log10 reduction from control* |
|---|---|
| LG-Cit 4 | 3.9 |
| LG-Cit 6 | 4.2 |
| O-Cit 2 | 1.5 |
| LGO Cit 6 | 6.4 |

*Log 10 reduction from control bacterial counts (control counts ranges from 1 × 10⁶ to 5 × 10⁶)

These data show that when citric acid was used in combination with 0.4% LG oil+0.2% O oil (LGO-Cit 6)superior antibacterial activity was observed as compared to that of combination of Citric acid and LG oil 0.6% (( LG-Cit 6) or the additive activity of Citric acid+0.4% LG oil( LG-Cit 4)and Citric acid+0.2% Orange oil (O-Cit 2).

### 16. EXAMPLE 12

Certain soaps described in Example 10 were tested for antimicrobial activity.

The following method was used. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. Soft soap® and Dial® soaps containing 0.15% triclosan was also tested similarly at the same time. The soap base without essential oils and citric acid containing the culture were used as controls. The results, showing 30 second kill activity, are shown in Table 13.

TABLE 13

| Soap | Log10 reduction from control* |
|---|---|
| LGO-Cit 6 | 6.4 |
| LGO-Cit 7 | 6.5 |
| CO-Cit 6 | 5.1 |
| CO-Cit 7 | 5.2 |
| BO-Cit 6 | 2.87 |
| CRO-Cit 6 | 4.57 |

*Log 10 reduction from control bacterial counts (ranges from 1 × 10⁶ to 3 × 10⁶.)

These data show that LGO-Cit Soaps were found to exhibit higher antibacterial activity compared to the other essential oil/citric acid combination soaps tested.

### 17. EXAMPLE 13

The following experiments were performed to evaluate the antibacterial activity of triclosan, LG oil, and combinations of triclosan and LG oil.

Patent application WO/2007/077573 by Mukhopadhyay et al describes an antimicrobial composition containing triclosan and an essential oil where the ratio of triclosan to the essential oil is 1:5 to 1:100 and the preferred ratio range is 1:10 to 1:90. In the example provided in United States Patent Application Publication No. 20050019431 by Modak et al., TC and essential oil at 1:1 ratio showed neither synergistic nor enhanced activity.

Triclosan (TC) is often used in personal care products at a concentration of 0.15-0.3%. In order to determine whether or not TC at this concentration would enhance the activity of essential oil at 0.4-0.7% which is the concentration used in various formulations described in this application, the antibacterial activity of soaps containing triclosan (“TC”); LG oil; or TC and LG oil at TC:LG weight ratios of 1:1.7 to 1:4.6 were evaluated.

To prepare the soaps, TC, LG oil or their combination were dissolved in SDA40 B alcohol and then added to Softsoap® (a formulation lacking triclosan), then diluted with water, where the amount of SDA40B alcohol used represented 5.5% of the final solution and the amount of Softsoap(g used represented 92% of the final solution. Soft Soap® was used as the control in this study.

The following method was used. A mixture of 0.1 ml of 10⁸ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 14.

TABLE 14

| Soap | Log10 reduction from control* |
|---|---|
| Softsoap ® + 0.15% TC | 0.70 |
| Softsoap ® + 0.3% TC | 0.81 |
| Softsoap ® + 0.5% LG oil | 0.76 |
| Softsoap ® + 0.7% LG oil | 0.75 |
| Softsoap ® + 0.15% TC + 0.5% LG oil | 0.74 |
| Softsoap ® + 0.15% TC + 0.7% LG oil | 0.92 |
| Softsoap ® + 0.3% TC + 0.5% LG oil | 0.77 |
| Softsoap ® + 0.3% TC + 0.7% LG oil | 0.77 |

*Log 10 reduction from control bacterial counts (ranges from 5.8 × 10⁷ to 6.4 × 10⁷ cfu)

These results indicate that no synergistic or enhanced effect was seen when triclosan was combined with LG oil at weight ratios falling within the range of 1:1.7 to 1:4.6.

### 18. EXAMPLE 14

The antibacterial activity of soaps containing 1) TC-LGO-Cit 6 at weight ratios within the range of between 1:3.3 and 1:4.7 (TC:LG) and between 1:1.4 and 1:2(LG:Citric acid) were evaluated against S. aureus. To prepare the soaps, triclosan/essential oil(s)/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.1 ml of 10⁷ cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 15.

TABLE 15

| | Log10 reduction |
|---|---|
| Soap | from control* |
| Softsoap ® | — |
| Softsoap ® + 0.15% TC | 0.24 |
| Softsoap ® + 1% citric acid | 1.49 |
| Softsoap ® + 0.15% TC + $$ % citric acid | 2.01 |
| Softsoap ® + 0.15% TC + 0.5% LG oil + 1% | 2.41 |
| citric acid | |
| Softsoap ® + 0.15% TC + 0.4% LG oil + 0.2% | 7.93 |
| Orange oil + 1% citric acid | |

*Log 10 reduction from control bacterial counts (ranges from 1 × 10⁶ to 5 × 10⁶ cfu).

The foregoing data show that citric acid was found to enhance the activity of triclosan, and that addition of LG oil+O oil to a combination of triclosan and citric acid further enhanced the effect.

### 19. EXAMPLE 15

The following experiments were performed to compare the antibacterial activity of combinations of (i) lemongrass oil citric acid+triclosan; (ii) lemongrass oil+citric acid; and (iii) cinnamon oil-citric acid+triclosan, all in a Softsoap® base.

To prepare the soaps, triclosan/essential oil/citric acid were dissolved in SDA40 B alcohol and added to Softsoap® (lacking triclosan) and diluted with water, so that the final concentration of alcohol was 5.5% and the final concentration of Softsoap® was 92 percent. A mixture of 0.lml of 10 cfu/ml of S. aureus culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 16.

TABLE 16

| | Log10 |
|---|---|
| | reduction |
| Soap | from control* |
| Softsoap ® + 0.15% TC | 0.7 |
| Softsoap ® + 0.15% TC + 0.4% LG oil + 0.2% | 7.93 |
| Orange oil + 1% citric acid | |
| Softsoap ® + 0.4% LG oil + 0.2% Orange oil + | 5.73 |
| 1% citric acid | |
| Softsoap ® + 0.15% TC + 0.4% C oil + 0.2% | 5.50 |
| Orange oil + 1% citric acid | |
| Softsoap ® + 0.4% C oil + 0.2% Orange Oil + | 4.39 |
| 1% citric acid | |

*Log 10 reduction from control bacterial counts (ranges from 6.4 × 10⁷ to 9.9 × 10⁷ cfu)

The above data demonstrate, among other things, that LGO-Cit+Triclosan was found to be more effective than LGO-Cit and CO-Cit+Triclosan.

### 20. EXAMPLE 16

The following experiments were performed to evaluate the effect of adding various essential oil combinations, citric acid (0.5-0.7%), and SDA 40 B alcohol (5.5%) to commercial triclosan-containing soaps such as Dial® Soap and Softsoap® containing 0.15% Triclosan (“Dial(t Soap-TC” and “Softsoap®—TC” respectively). A mixture of 0.1 ml of 10⁸ cfu/ml of S. aureus culture and 0.1 ml of bovine serum were placed in a sterile culture tube. 0.8 ml of the test soap formulation (or phosphate buffered saline as control) was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 17.

Dial® Soap TC-CO-Cit 7

| Ingredients | W/W |
|---|---|
| Cinnamon oil | 0.5 |
| OrangeOil | 0.2 |
| Citric acid | 1.0 |
| SDA 40 B alcohol | 5.5 |
| Dial ® Soap-TC | 92.8 |

Dial® Soap TC-LGO-Cit 7

| Ingredients | W/W |
|---|---|
| Lemon Grass oil | 0.5 |
| Orange Oil | 0.2 |
| Citric acid | 1.0 |
| SDA 40 B alcohol | 5.5 |
| Dial Soap ®-TC | 92.8 |

Dial® SoapTC-LG-Cit 5

| Ingredients | W/W |
|---|---|
| Lemon Grass oil | 0.5 |
| Citric acid | 1.0 |
| SDA 40 B alcohol | 5.5 |
| Dial ® Soap-TC | 92.8 |

Softsoap® TC-LGO-Cit 7

| Ingredients | W/W |
|---|---|
| Lemon Grass oil | 0.5 |
| OrangeOil | 0.2 |
| Citric acid | 1.0 |
| SDA 40 B alcohol | 5.5 |
| Softsoap ®-TC | 92.7 |

TABLE 17

| | Log10 |
|---|---|
| | reduction |
| Soap | from control* |
| Dial ® Soap TC | 0.36 |
| Dial ® Soap-TC-CO-Cit 7 | 3.9 |
| Dial ® Soap-TC-LG-Cit 5 | 3.35 |
| Dial ® Soap-TC-LGO-Cit 7 | 5.09 |
| Softsoap ®-TC | 0.33 |
| Softsoap ®-TC-LGO-Cit 7 | 4.66 |
| Softsoap ®-TC + 1% citric acid | 2.64 |

*Log reduction from control bacterial counts (ranges from 2 0 × 10⁸ to 3.5 × 10⁸ cfu)

The above results indicate that citric acid was found to enhance the activity of soaps containing triclosan; the combination of citric acid and essential oils was found to increase the antimicrobial activity of soap containing triclosan, and superior antimicrobial action was associated with a combination of citric acid, lemongrass and orange oils, and triclosan.

### 21. EXAMPLE 17

The pH of soaps containing 1% citric acid typically ranges between 3.2-3.3. To determine whether or not the superior efficacy observed with the combination of essential oils and citric acid is due to the acidic pH, certain EO/citric acid containing-soaps were adjusted to pH 6.0 with 10 N sodium hydroxide and their antibacterial efficacy tested and compared to the corresponding soaps without pH adjustment. For the evaluation of antimicrobial activity, a mixture of 0.1 ml of 10⁷ cfu/ml of S. aureus culture (ATCC #6538) and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results are shown in Table 18. (“Softsoap®13 TC” is Softsoap® containing 0.15 percent triclosan).

TABLE 18

| | Log10 |
|---|---|
| | reduction |
| Soap | from control* |
| CO-Cit + Softsoap ® - TC pH 3.25 | 3.9 |
| CO-Cit + Softsoap ® - TC pH 6.0 | 3.25 |
| CLGO-Cit + Softsoap ® - TC pH 3.25 | 5.1 |
| CLGO-Cit + Softsoap ® - TC pH 6.0 | 5.65 |

*Log 10 reduction from control bacterial counts (ranges from 1 × 106 to 5 × 106.)

Conclusion: The efficacy was similar at both pH values tested. This indicates that the superior activity of essential oils and citric acid observed is not due to the acidic pH.

### 22. EXAMPLE 18

Household cleansers were prepared comprising citric acid (1-2%), alcohol, and either (i) lemongrass oil; (ii) a combination of lemongrass oil and pine oil; (iii) a combination of lemongrass oil and orange oil; or (iv)a combination of pine oil and orange oil. The antimicrobial effectiveness of these formulations were tested and compared to commercial Pinesol® cleanser (containing 8.7 percent pine oil and other ingredients including detergent and other cleaning agents) as a control.

Stock solution of hard surface Disinfectant-LG-Cit 2:

| Ingredients | % (W/W) |
|---|---|
| Lemongrass oil | 2.0 |
| Citric Acid | 20.0 |
| SDA 40B alcohol | 76.5 |
| Pluronic Surfactant L-61 | 0.5 |
| Pluronic Surfactant F-127 | 0.5 |
| Pluronic Surfactant F-87 | 0.5 |

Stock solution of hard surface Disinfectant.-LGP-Cit 4

| Ingredients | % W/W |
|---|---|
| Lemongrass oil | 1.0 |
| Pine oil | 3.0 |
| Citric Acid | 20 |
| SDA 40B alcohol | 74.5 |
| Pluronic Surfactant L-61 | 0.5 |
| Pluronic Surfactant F-127 | 0.5 |
| Pluronic Surfactant F-87 | 0.5 |

Stock solution of hard surface Disinfectant.-P-Cit 5

| Ingredients | % W/W |
|---|---|
| Pine oil | 5.0 |
| Citric acid | 20 |
| SDA 40B alcohol | 73.5 |
| Pluronic Surfactant L-61 | 0.5 |
| Pluronic Surfactant F-127 | 0.5 |
| Pluronic Surfactant F-87 | 0.5 |

After tenfold dilution of each stock solution the disinfectant contained the following percentages (w/w) of each ingredient:

Surface Disinfectant-LG cit 2
0.2% Lemon grass oil+2% Citric acid+7.65% Alcohol+0.15% surfactants

Surface Disinfectant-LG P cit 4
0.3% Pine oil+0.1% Lemon grass oil+2% Citric Acid+7.45% alcohol+0.15% Surfactants

Surface Disinfectant-P cit 5
0.5% Pine oil+2% Citric acid+7.45% alcohol+0.15% surfactants. To prepare the solution of Pinesol® to serve as control, as per the manufacturer's instruction, 6 ml of the Pinesol® containing 8.5% pine oil was diluted to 100 ml. This diluted sample contained 0.52% pine oil.

To test the antimicrobial activity, 0.1 ml of culture containing approximately 1×10⁷ colony forming units (“cfu”) of S. aureus per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 19

| | Log₁₀ reduction from control bacterial counts* | | | |
|---|---|---|---|---|
| | Disinfectant | Disinfectant | Disinfectant | |
| Organism | LG-Cit | LGP-Cit | P-Cit | Pine Sol |
| S. aureus | 3.56 | 1.89 | 0.81 | 2.4 |

*Log 10 reduction from control bacterial counts (ranges from 1 × 10⁶-5 × 10⁶)

These data indicate that a surface cleaner containing 0.2% LG oil and 2.0% Citric acid was found to be considerably more effective than a cleaner containing 0.5% Pine oil and 2% Citric acid as well as commercial Pinesol® Surface cleaner containing 0.52% Pine oil . The cleanser containing 0.3% Pine oil+0.1% LG oil+2% Citric acid was also found to be more effective than the one containing 0.5% Pine oil and 2% Citric acid.

### 23. EXAMPLE 19

The following stock solution was prepared:

Stock Solution of hard surface Disinfectant.-POCit 7

| Ingredient | % W/W |
|---|---|
| Pine oil | 5.0 |
| Orange oil | 2.0 |
| Citric acid | 10 |
| SDA 40B alcohol | 53.5. |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. This diluted samples contained the following concentrations of active ingredients:

### Surface Disinfectant-PO Cit 7

0.5% Pine oil+0.2% Orange oil+1% Citric Acid+5.35% alcohol+0.15% Surfactants

The following stock solution was prepared:

Stock Solution of hard surface Disinfectant.-LGOCit 7

| Ingredient | % W/W |
|---|---|
| Lemongrass oil | 5.0 |
| Orange oil | 2.0 |
| Citric acid | 10 |
| SDA 40B alcohol | 53.5. |
| Pluronic Surfactant L-61 | 0.5 |
| Pluronic Surfactant F-127 | 0.5 |
| Pluronic Surfactant F-87 | 0.5 |

7.2% of the stock hard disinfectant was diluted with water to 100% before use. This diluted samples contained the following concentrations of active ingredients:

### Surface Disinfectant-LGO Cit 7

0.5% LG oil+0.2% Orange oil+1% Citric Acid+5.35% alcohol+0.15% Surfactants The mthod used in Example 18 was used to test antimicrobial activity.

TABLE 20

| | S. aureus | P. aeruginosa | E. coli |
|---|---|---|---|
| Log 10 reduction in | 0.6 | 5.1 | 5.1 |
| bacteria-PO-Cit 7 | | | |
| Log10 reduction in | 5.9 | 4.8 | 5.09 |
| bacteria-LGO-Cit 7 | | | |

*Log₁₀ reduction from control bacterial counts (ranges from 1 × 10⁶-5 × 10⁶)

The foregoing data indicate that LGO-Cit is effective against both gram positive and gram negative organisms while PO Cit is not very effective against the Gram positive organism S aureus.

### 24. EXAMPLE 20

The following experiments were carried out using either soap or surface disinfectants containing the EO(s)/citric acid combinations indicated. The test organism used was Candida albicans.

Where soap was employed, the following method was used. A mixture of 0.1 ml of 10⁷ cfu/ml of C. albicans culture and 0.1 ml of bovine serum were placed in a sterile culture tube. 0.8 ml of the test soap formulation was added to the tube and vortexed for 30 seconds. 9.0 ml DFN was added to the tube to neutralize the activity of the soap; this tube was then vortexed and serially diluted with DFN. 0.5 ml of the diluted solution was plated on trypticase soy agar plates, incubated at 37° C. for 24-48 hrs and the colony counts were determined. The results, showing 30 second kill activity, are shown in Table 21.

Where surface disinfectant was employed, the following method was used. 0.1 ml of culture containing approximately 1×10⁷ colony forming units (“cfu”) of C. albicans per milliliter was spread evenly on the surface of 2.5×11 cm² tiles using a glass rod and left at room temperature for 10 minutes to dry. After 10 minutes 0.3 ml of the diluted surface disinfectant was spread evenly on the tiles with a sterile glass rod and left for another 10 minutes to dry. The tiles were rinsed with 9.6 ml of inactivating medium (BPBNS), which was collected for testing. The collected medium was serially diluted and 0.5 ml was plated onto TSA plates and incubated at 37° C. for 18-24 hours. The colonies on the plates were counted and the values converted to log₁₀.

TABLE 21

| | Log10 |
|---|---|
| | reduction |
| Formulation | from control* |
| CO-Cit 6 Soap | 1.02 |
| LGO-Cit 6 Soap | 1.27 |
| CO-Cit 7 Surface Disinfectant | 5.2 |
| LGO-Cit 7 Surface Disinfectant | 4.81 |

*Control counts range from 1 × 10⁶ to 5 × 10⁶

These results show that CO groups and LGO groups show similar activity against C. albicans.

Various patent and non-patent publications are cited herein, the contents of which are hereby incorporated by reference in their entireties.

We claim:

1. A personal care product composition comprising (i) one or more essential oil or individual constituent thereof at a concentration between about 0.1 and 1.2 percent (weight/weight); and (ii) a fruit acid at a concentration between about 0.5 and 1.5 percent (weight/weight); having one or more characteristic selected from the group consisting of:
(a) the composition further comprises one or more than one component selected from the group consisting of emollients, thickening agents, humectants, silicone polymers, and hydrogels;
(b) the composition is suitable for use as a personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream; and
(c) regular exposure of the skin to the composition does not produce skin irritation in a normal subject.

2. The personal care product composition of claim 1, wherein (i) the one or more essential oil or individual constituent thereof is present at a concentration between about 0.1 and 1 percent (weight/weight); (ii) the fruit acid is present at a concentration between about 0.125 and 1 percent (weight/weight); and further comprising alcohol at a concentration between about 5 and 20 percent (weight/weight).

3. The personal care product composition of claim 2, further comprising triclosan at a concentration between about 0.05 and 1 percent (weight/weight).

4. The personal care product composition of claim 1, 2 or 3 wherein the essential oil and/or constituent thereof is selected from the group consisting of lemongrass oil, an individual constituent of lemongrass oil, orange oil, an individual constituent of orange oil, cinnamon leaf oil, and individual constituent of cinnamon leaf oil, basil oil, and an individual constituent of basil oil.

5. The personal care product composition of claim 1, 2 or 3 wherein the fruit acid is citric acid.

6. The personal care product composition of claim 4 wherein the fruit acid is citric acid.

7. A personal care product composition comprising:
(i) lemograss oil or an individual constituent thereof;
(ii) orange oil or an individual constituent thereof;
(iii) fruit acid at a concentration between about 0.25 and 1 percent (weight/weight); and
(iv) alcohol at a concentration between about 5 and 20 percent (weight/weight), wherein the total concentration of lemon grass oil or an individual constituent thereof and orange oil or an individual constituent thereof is between about 0.2 and 0.7 percent (weight/weight).

8. The personal care product composition of claim 6, further comprising triclosan at a concentration between about 0.05 and 1 percent.

9. The personal care product composition of claim 7 or 8, wherein the fruit acid is citric acid.

10. The personal care product composition of claim 7 or 8 which is suitable for use as a personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream.

11. The personal care product composition of claim 9 which is suitable for use as a personal care product selected from the group consisting of a bar soap, a liquid hand soap, a hand sanitizer, a body wash, an acne treatment, a shampoo, a hair conditioner, a cosmetic, a deodorant, a body lotion, a hand cream, a topical cream, an aftershave lotion, a skin toner, a mouth wash, a toothpaste, a sunscreen lotion, a baby cleansing wipe, and a diaper cream.

12. A surface cleaner composition comprising more than one essential oil or constituent thereof at a total concentration between about 0.1 and 1 percent (weight/weight), a fruit acid at a concentration of between about 1 and 2 percent (weight/weight), and an alcohol, at a concetration of between about 5 and 20 percent (weight/weight), where the essential oils or constituents thereof are selected from the group consisting of lemongrass oil, an individual constituent of lemongrass oil, orange oil, and individual constituent of orange oil, basil oil, an individual constituent of basil oil, pine oil, and an individual constituent of pine oil.

13. The surface cleaner composition of claim 12, further comprising a surfactant.

14. The surface cleaner composition of claim 12, comprising pine oil and orange oil.

15. The surface cleaner composition of claim 12, comprising pine oil and lemongrass oil.

16. The surface cleaner composition of claim 12, comprising lemongrass oil and orange oil.

17. A veterinary product composition comprising (i) one or more essential oil or individual constituent thereof at a concentration between about 0.1 and 1.2 percent (weight/weight); and (ii) a fruit acid at a concentration between about 0.5 and 1.5 percent (weight/weight); wherein the veterinary product composition is suitable for use as a product selected from the group consisting of a pet shampoo, a pet cleansing wipe, an ear cleaning liquid, a cage cleaner, a topical cream, a teat dip, or a pet body splash.
